(19) United States
(12) Patent Application Publication
(10) Pub. No.: US 2016/0022591 A1
(43) Pub. Date: Jan. 28, 2016

(54) EXTENDED RELEASE PHARMACEUTICAL SOLID DOSAGE FORMULATIONS

(71) Applicant: MYLAN INC., Morgantown, WV (US)

(72) Inventors: John David KIRSCH, Waynesburg, PA (US); Chaoju XIAO, Morgantown, WV (US)

(21) Appl. No.: 14/775,221

(22) PCT Filed: Mar. 13, 2014
(86) PCT No.: PCT/US14/26464

§ 371 Date: Sep. 11, 2015

Related U.S. Application Data
(60) Provisional application No. 61/791,262, filed on Mar. 15, 2013.

(51) Int. Cl. A61K 9/28; A61K 31/165; A61K 9/20

(52) U.S. Cl. CPC A61K 9/2866; A61K 9/2095; A61K 31/165; A61K 9/2893; A61K 9/2846

(57) ABSTRACT

A solid pharmaceutical dosage form, e.g., a tablet, is disclosed which comprises (a) a sustained released matrix core containing i) an at least sparingly water soluble active pharmaceutical ingredient, ii) a hydrophilic polymer, iii) an optional hydrophobic polymer, iv) an optional wax component, and v) optional diluent; and (b) a semi-permeable functional film coating surrounding the core, wherein the dosage form is substantially resistant to dose dumping when administered in the presence of ethanol. A method for making the dosage form is also provided in which the dosage form is coated with a semi-permeable coating.

### FIELD OF THE INVENTION

The present disclosure relates to extended release pharmaceutical solid dosage formulations, such as tablets or caplets, which are resistant to burst release, even in the presence of ethanol, and which contain an extended release matrix core and a semi-permeable functional coating.

### BACKGROUND

U.S. Pat. No. 4,882,167 to Jang, incorporated herein by reference in its entirety, discloses a controlled and continuous release matrix for tablets or implants of biologically active agents comprising a hydrophobic carbohydrate polymer, e.g. ethyl cellulose, and at least one digestive-difficult soluble component, i.e. a wax, e.g. carnauba wax, fatty acid material or neutral lipid.

U.S. Pat. No. 6,254,887 to Miller et al, incorporated herein by reference in its entirety, discloses a controlled release oral pharmaceutical tablet comprising tramadol for dosing every 24 hours. The tablet is coated with a controlled release coating comprising water insoluble wax, water insoluble polymer, water insoluble cellulose and mixtures thereof.

U.S. Pat. Appl. Pub. No. 2008/0057123 to Grenier et al, incorporated herein by reference in its entirety, discloses controlled release oral dosage formulations containing one or more active agents, in the form of a tablet containing a core or central layer and one or more barrier layers. The core may contain one or more enteric materials or polymeric materials which modulates the release of the active agent. The core may also contain non-enteric polymeric materials, including hydrophilic polymers such as crosslinked polyvinylpyrrolidone, hydroxypropylmethylcellulose, hydroxypropylcellulose, crosslinked sodium carboxymethylcellulose, carboxymethyl starch, acrylic and hydrophobic polymers such as methacrylic acid polymers and copolymers, polyesters, polyanhydrides, polymethylvinylether/anhydride copolymers, potassium methacrylate-divinylbenzene copolymer, polyvinylalcohols, glucan, scleroglucan, mannan, starch and derivatives thereof, betacyclodextrins, cyclodextrin derivatives containing linear and/or branched polymeric chains, and combinations thereof.

U.S. Pat. Appl. Pub. No. 2006/0228415 to Oberegger et al, incorporated herein by reference in its entirety, discloses a modified-release tablet containing water soluble bupropion hydrochloride comprising (i) a core comprising an effective amount of bupropion hydrochloride, a binder, a lubricant; (ii) a control releasing coat surrounding said core; and (iii) a moisture barrier surrounding said control releasing coat. In some embodiments, the moisture barrier does not function as an enteric coating as defined by a USP test which requires for an enteric layer-coated tablet, when placed in 0.1N HCl for one hour, that the total amount of the drug released from the core does not exceed 10% and not less than 75% of the drug is released at 45 minutes in pH 6.8 buffer. The moisture barrier may contain an enteric polymer such as a methacrylic acid copolymer, Eudragit L 30 D-55. The core may also contain a binder selected from the group consisting of modified starch, gelatin, polyvinylpyrrolidone, cellulose derivatives, polyvinyl alcohol and any combination thereof.

U.S. Pat. No. 5,854,290 to Arnsten et al, incorporated herein by reference in its entirety, discloses treating behavioral disinhibition such as Attention-Deficit Hyperactivity Disorder, by administering guanfacine through intramuscular injection.

U.S. Pat. No. 6,287,599 to Burnside et al. discloses sustained release pharmaceutical dosage forms with minimized pH dependent dissolution profiles or pH independent dissolution profiles. The dosage forms comprise (a) at least one pharmaceutically active agent that is pH dependent, such as guanfacine hydrochloride, propanolol, and metoprolol, (b) at least one non-pH dependent sustained release agent, and (c) at least one pH dependent agent that increases the rate of release of said at least one pharmaceutically active agent from the tablet at a pH in excess of 5.5. The non-pH dependent sustained release agent is selected from the group consisting of ethylcellulose, cellulose acetate, vinyl acetate/vinyl chloride copolymers, acrylate/methacrylate copolymers, polyethylene oxide, hydroxypropyl methylcellulose, carageenan, alginic acid and salts thereof, hydroxyethyl cellulose, hydroxypropyl cellulose, karaya gum, acacia gum, tragacanth gum, locust bean gum, guar gum, sodium carboxymethyl cellulose, methyl cellulose, beeswax, carnauba wax, cetyl alcohol, hydrogenated vegetable oils, and stearyl alcohol. The pH dependent agent can be an enteric agent selected from cellulose acetate phthalate, hydroxypropyl methylcellulose phthalate, polyvinyl acetate phthalate, methacrylic acid copolymers, cellulose acetate trimellitate, hydroxypropyl methylcellulose acetate, succinate, shellac, and zein.

U.S. Pat. No. 6,811,794 to Burnside et al. discloses sustained release pharmaceutical dosage forms with minimized pH dependent dissolution profiles or pH independent dissolution profiles. In particular, the reference teaches a pharmaceutical composition, comprising: (a) guanfacine hydrochloride; (b) hydroxypropyl methylcellulose; (c) ammonio methacrylate copolymer; (d) microcrystalline cellulose; (e) a methacrylic acid copolymer; (f) glyceryl behenate (a wax); (g) fumaric acid; (h) lactose monohydrate; (i) povidone; and (j) crospovidone granulated blend.

C. Hsiao and R. K. Chang, “Eudragit RL and RS Pseudo lattices: properties and performance in pharmaceutical coating as a controlled release membrane for theophylline pellets,” Drug Dev. Ind. Pharm. 15(2), 1870196 (1989) disclose that acrylic coatings of theophylline pellets containing varied ratios of high cation density Eudragit RL 30 and lower cation density Eudragit RS 30D coatings had no significant effect on drug release profiles.

M. M. Kanakal, et al., “Effect of Coating Solvent Ratio on the Drug Release Lag Time of Coated Theophylline Osmotic Tablets, Tropical Journal of Pharmaceutical Research, June 2009; 8 (3): 239-245, investigated the effect of hydro-alcohol coating solvent ratio on the surface texture and lag time of porous theophylline osmotic tablets formulated by direct compression and coated by spraying with varying ratios of water-alcohol containing hydroxypropyl methylcellulose (HPMC, 5 cps) as primary swelling layer and Eudragit(r) RSPO and RLPO as porous layer. The viscosity of HPMC coating solution was determined using Brookfield viscometer. Surface morphology was investigated with scanning electron microscopy (SEM). In vitro drug release studies to assess lag time were performed in simulated gastric fluid (SGF) and simulated intestinal fluid (SIF). No significant change in the viscosity of HPMC coating solution was found when water-alcohol solvent ratio was varied. SEM revealed profound distortions in coating texture with a high proportion of either solvent in the coating solution. Increasing the amount of either water or alcohol in the coating solution increased the roughness of coated surface which contributed to a burst release of drug due to early opening of the tablet as a result of the high osmotic pressure and low mechanical strength of the coated layer. The optimum coating solvent ratio for spray-coating the osmotic tablets was watenalcohol (60:34) and it resulted in a coating with smooth texture and which successfully modulated drug release lag time of the coated tablets. The optimum ratio of water/alcohol (60:34) as coating solvent produced a smooth coated tablet surface texture and modulated the drug release lag time of the tablets.

Typical matrix core tablets will have high initial burst release within one hour after administration to a patient, with 20% or more of the active ingredient being released. Such high release rate typically results from large surface area as well as delayed gelling of the matrix, or slow polymer hydration rate. Providing the matrix with a functional coating can, in some instances, reduce the initial release rate of the active ingredient. However, such coated tablets, where their coating is soluble in alcohol, are susceptible to abuse by the patient, resulting in “alcohol dumping” of the active ingredient when taken in conjunction with ethanol.

Han Lennernas, “Ethanol-Drug Absorption Interaction: Potential for a Significant Effect on the Plasma Pharmacokinetics of Ethanol Vulnerable Formulations,”

Mol. Pharmaceutics, 2009, 6 (5), pp. 1429-1440, states that when ethanol interacts with an oral controlled release product, such that the mechanism controlling drug release is impaired, the delivery of the dissolved dose into the small intestine and the consequent absorption may result in dangerously high plasma levels. Thus, a pharmacokinetic ethanol-drug interaction is a very serious safety concern when substantially the entire dose from a controlled release product is rapidly emptied into the small intestine (dose dumping), having been largely dissolved in a strong alcoholic beverage in the stomach during a sufficient lag-time in gastric emptying.

### SUMMARY

The present disclosure provides a matrix core tablet containing an at least sparingly water soluble active pharmaceutical ingredient, which tablet is not susceptible to high initial burst release when administered to a patient, even if co-administered with ethanol. Moreover, the disclosure provides a matrix core tablet coated with a functional coating which slows down initial drug release by limiting the drug exposure to the medium encountered upon administration, e.g., for up to about two hours after administering to the patient. The present disclosure also provides a solid dosage form which is coated with a semi-permeable functional film coating that slows down initial drug release by limiting exposure of a drug to the medium encountered upon administration and which has little impact on overall drug release profile controlled by the matrix core which itself is resistant to alcohol dose dumping. The coating can be desirably short-lived upon administration to a patient, e.g. no greater than about one or two hours in the medium first encountered upon ingestion, such as the stomach contents of a patient. The present disclosure also can provide a solid dosage formulation which can be made using common ingredients on readily available equipment, and which provides bioequivalent results compared to branded products.

In one aspect, the present disclosure relates to a solid pharmaceutical dosage form comprising: (a) a sustained released matrix core containing i) an at least sparingly water soluble active pharmaceutical ingredient, ii) a hydrophilic polymer, iii) an optional hydrophobic polymer, iv) an optional wax component, and v) an optional diluent; and (b) a semi-permeable functional film coating surrounding the core, wherein the dosage form is substantially resistant to dose dumping when administered in the presence of ethanol.

In an embodiment of this aspect, the semi-permeable functional film coating is applied to the core as an aqueous dispersion.

In another embodiment of this aspect of the disclosure, the film coating surrounding the core is substantially insoluble in ethanol.

In certain embodiments of this aspect of the disclosure, the film coating surrounding the core comprises i) high permeability cationic copolymer derived from esters of acrylic acid and methacrylic acid; and ii) low permeability cationic copolymer derived from esters of acrylic acid and methacrylic acid. The cationic copolymers can contain alkaline groups, e.g., the cationic copolymers are ammonioalkyl methacrylate copolymers. In some embodiments, the weight ratio of i) high permeability cationic copolymer derived from esters of acrylic acid and methacrylic acid to ii) low permeability cationic copolymer derived from esters of acrylic acid and methacrylic acid ranges from about 0.1 to about 10, e.g., from about 0.4 to about 0.6. In some embodiments the alkaline groups are selected from alkali metal hydroxides, alkaline earth metal hydroxides, and aluminum hydroxide, in particular NaOH, KOH, Ca(OH)2, Mg(OH)2, Al(OH)3, in any pharmaceutically acceptable form, for example in form of hydrate(s) or solvate(s) thereof, in crystalline or non-crystalline form(s), such as amorphous form(s).

In some embodiments of this aspect of the invention, the film coating surrounding the core comprises a plasticizer. In certain embodiments, the plasticizer component is selected from at least one of the group consisting of triethyl citrate, triacetin, ethylene vinyl acetate, polyethylene glycol 200 to 8000, glyceryl monostearate, polyvinyl pyrrolidone, dibutyl phthalate, dibutyl sebacate, fractionated coconut oil, and methylparaben. In certain embodiments, the plasticizer component comprises triethyl citrate.

In still other embodiments of this aspect of the disclosure, the film coating surrounding the core comprises an antiadherent. In certain embodiments, the antiadherent is selected from at least one of glyceryl monostearate, polysorbate 80, and talc. In some embodiments, the antiadherent comprises talc.

In yet other embodiments of this aspect of the disclosure, the film coating surrounding the core comprises ethylcellulose polymer. In certain embodiments, the film coating further comprises cellulose ether polymer. The cellulose ether polymer can be selected from at least one of the group consisting of hydroxypropylmethylcellulose, hydroxypropylcellulose and methylcellulose. Clear Opadry®, available from Colorcon, comprises hydroxypropylmethylcellulose and polyethylene glycol and is a suitable component for film coating of the present disclosure.

In some embodiments of this aspect of the disclosure, the disclosure relates to the dosage form wherein i) the hydrophilic polymer is selected from at least one of the group consisting of polyvinylpyrrolidone, hydroxypropylmethylcellulose, hydroxypropyl cellulose, polyethylene oxide, and xanthan gum, ii) the optional hydrophobic polymer is selected from at least one of the group consisting of ethylcellulose, cellulose acetate, cellulose acetate butyrate, methacrylic acid copolymer, and chitosan, iii) the optional wax is selected from at least one of the group consisting of glyceryl behenate, carnauba wax, fatty alcohols, and hydrogenated vegetable oil, and iv) the optional diluent is selected from at least one of the group consisting of lactose, microcrystalline cellulose, magnesium stearate, colloidal silica, calcium phosphate dibasic, calcium sulfate, sucrose, and mannitol.

In other embodiments, the disclosure relates to a dosage form wherein i) the hydrophilic polymer is selected from at least one of the group consisting of polyvinylpyrrolidone and hydroxypropylmethylcellulose, ii) the hydrophobic polymer is selected from at least one of the group consisting of ethylcellulose, cellulose acetate, cellulose acetate butyrate, methacrylic acid copolymer, and chitosan; iii) the wax comprises glyceryl behenate, and iv) the optional diluent is selected from at least one of the group consisting of lactose, microcrystalline cellulose, magnesium stearate, colloidal silica, and crospovidone.

In certain embodiments of this aspect of the disclosure, the disclosure relates to a dosage form wherein the semi-permeable functional film coating surrounding the core ranges from about 5 to about 15 wt. % of the core, e.g., about 10 wt. % of the core.

In various embodiments of this aspect of the disclosure, the disclosure relates to a dosage form wherein the at least sparingly water soluble active pharmaceutical ingredient is selected from at least one of the group consisting of guanfacine, metoprolol, ropinirole, propranolol, amphetamine, Zolpidem, and pharmaceutically acceptable salts thereof. In certain embodiments, the at least sparingly water soluble active pharmaceutical ingredient comprises guanfacine. In various embodiments, the at least sparingly water soluble active pharmaceutical ingredient comprises the hydrochloride salt of guanfacine.

In some embodiments of this aspect of the disclosure, the semi-permeable functional film coating surrounding the core is substantially absent one hour after administration of the dosage form to a patient. In certain embodiments, the semi-permeable functional film coating surrounding the core is substantially absent two hours after administration of the dosage form to a patient.

In various embodiments of this aspect of the disclosure, the dosage form is suitable for oral administration in a patient.

In another aspect of the disclosure, the disclosure relates to a method for preparing a solid pharmaceutical dosage form comprising: i) compacting a mixture comprising at least sparingly water soluble active pharmaceutical ingredient, hydroxypropylmethylcellulose, microcrystalline cellulose, lactose, and glyceryl behenate to provide a compacted first blend; ii) milling the compacted first blend to provide a milled first blend; iii) mixing the milled first blend with a second blend comprising polyvinylpyrrolidone, crospovidone, lactose, optional glyceryl behenate, colloidal silica, magnesium stearate, and colorant to provide a third blend; iv) compressing the third blend into core tablets; and v) applying a semifunctional film coating to the core tablets as an aqueous dispersion; to provide coated tablets substantially resistant to dose dumping when administered in the presence of ethanol.

In some embodiments of this aspect of the disclosure, the at least sparingly water soluble active pharmaceutical ingredient is selected from at least one of the group consisting of guanfacine, metoprolol, ropinirole, propranolol, amphetamine, Zolpidem, and pharmaceutically acceptable salts thereof.

In certain embodiments of this aspect of the disclosure, the film coating comprises i) high permeability cationic copolymer derived from esters of acrylic acid and methacrylic acid; and ii) low permeability cationic copolymer derived from esters of acrylic acid and methacrylic acid.

In some embodiments of this aspect of the disclosure, the film coating comprises ethylcellulose polymer.

In another aspect, the disclosure relates to a method for preparing a solid pharmaceutical dosage which comprises i) compacting a mixture comprising hydrochloride salt of guanfacine, hydroxypropylmethylcellulose, microcrystalline cellulose, lactose, and glyceryl behenate to provide a compacted first blend; ii) milling the compacted first blend to provide a milled first blend; iii) mixing the milled first blend with a second blend comprising polyvinylpyrrolidone, crospovidone, lactose, optional glyceryl behenate, colloidal silica, magnesium stearate, and colorant to provide a third blend; iv) compressing the third blend into core tablets; v) coating the core tablets with a semifunctional film coating applied as an aqueous dispersion wherein the film coating comprises high permeability cationic copolymer derived from esters of acrylic acid and methacrylic acid and low permeability cationic copolymer derived from esters of acrylic acid and methacrylic acid, or in the alternative, the film coating comprises ethylcellulose polymer; and vi) drying the coated tablets to remove water therefrom; wherein the resulting dosage form is substantially resistant to dose dumping when administered in the presence of ethanol.

### DETAILED DESCRIPTION

The present disclosure provides a solid pharmaceutical dosage form comprising: (a) a sustained released matrix core containing i) an at least sparingly water soluble active pharmaceutical ingredient, ii) a hydrophilic polymer, iii) an optional hydrophobic polymer, iv) an optional wax component, and v) optional diluent; and (b) a semi-permeable functional film coating surrounding the core, wherein the dosage form is substantially resistant to dose dumping when administered in the presence of ethanol.

For present purposes, the term “solid pharmaceutical dosage form” encompasses, but is not restricted to, pharmaceutical compositions being in the solid state at least at room temperature (about 23° C.). The solid pharmaceutical dosage form according to the present disclosure can be present in the form of tablets, pills, powders, lozenges, sachets, capsules, in particular soft and hard gelatine capsules, (filled with powders, granules or pellets), granules or pellets comprising or consisting of a composition comprising guanfacine or guanfacine hydrochloride. Preferably, the dosage form is suitable for oral application.

Sustained-release matrix core systems include, for our purposes, any drug delivery system that achieves slow release of drug from the matrix core over an extended period of time. If the system is successful at maintaining constant drug levels in the blood or target tissue, it is considered a controlled-release system. If it is unsuccessful at this but nevertheless extends the duration of action over that achieved by conventional delivery, it is considered a prolonged-release system.

Pharmaceutically active agents or active pharmaceutical ingredients (APIs) which may be included in the composition of the disclosure include, but are not limited to, at least sparingly water soluble drugs. For present purposes “at least sparingly water soluble” means at least about 10 mg of the drug can be completely dissolved in one mL of water at 25° C. Suitable drugs for use in the present disclosure include sparingly soluble drugs (10-50 mg/mL at 25° C.), soluble drugs (50-100 mg/mL at 25° C.), freely soluble drugs (100-1000 mg/mL at 25° C.), and very soluble drugs (>1000 mg/mL at 25° C.). Such drugs include, but are not limited to, guanfacine hydrochloride, ropinirole, Zolpidem, guanadrel sulfate, reserpine, anagrelide hydrochloride, propanolol, metoprolol, atenolol, timolol, erthyrthromycin, clonidine, chlorpheniramine, bromopheniramine, diltiazen, and scopolamine. In general, the pharmaceutically active agent is present in the composition in an amount of from about 0.1 wt. % to about 70 wt. %, preferably from about 1 wt. % to about 40 wt %. It is to be understood that the scope of the present invention is not to be limited to any particular pharmaceutically active agent.

Preferable average particle size of active ingredient of the present disclosure, in particular the preferable average particle size of particles consisting of or comprising guanfacine or guanfacine hydrochloride, depends on the technological process used. In case of a wet granulation process, preferable average particle size of the active ingredient, in particular the preferable average particle size of particles consisting of or comprising guanfacine or guanfacine hydrochloride, can be from about 2 μm to about 250 μm. In case of direct compression, preferable average particle size, in particular the preferable average particle size of particles consisting of or comprising guanfacine or guanfacine hydrochloride, can be less than about 150 μm.

The term “average particle size” as used herein refers to the volume mean diameter of particles. The diameter and volume mean diameter can be determined by laser light scattering using e.g. a Malvern-Mastersizer Apparatus MS 2000. Particle sizes are determined by measuring the angular distribution of laser light scattered by a homogeneous suspension of particles. The particles to be subjected to the particle size measurement are first suspended in appropriate non-polar dispersant and then subjected to a size determination in a Malvern Mastersizer MS 2000 instrument. Usually, 100-800 mg of substance are dispersed in 5-10 ml of dispersant. In particular, 100 mg of substance can be dispersed in 10 ml of vegetable oil at a room temperature.

The amount of the active ingredient guanfacine or guanfacine hydrochloride present in the solid pharmaceutical composition according to the present invention can be from about 0.5 to about 20 mg, preferably from about 1 to about 8 mg and most preferably from about 1 to about 4 mg of guanfacine or guanfacine hydrochloride per final dosage form, for example, per tablet.

By “substantially resistant to dose dumping when administered in the presence of ethanol” is meant that the dosage form retains its sustained release properties even when exposed to high concentrations of ethanol. For example, the release rate of the dosage form in an ethanol concentration of at least 4%, preferably 20%, more preferably 40% is substantially similar to the release rate of the dosage form in an ethanol-free environment. Han Lennernas, “Ethanol-Drug Absorption Interaction: Potential for a Significant Effect on the Plasma Pharmacokinetics of Ethanol Vulnerable Formulations,” Mol. Pharmaceutics, 2009, 6 (5), pp 1429-1440, incorporated by reference in its entirety, recommends a two hour time frame for screening the in vitro dissolution profile of a controlled-release product in ethanol concentrations of up to 40%. Therefore, one definition for resistance to alcohol induced dose dumping is a dosage form that releases less than 60% of the active agent after 30 minutes in an ethanol concentration of at least 40%, less than 70% of the active agent after 60 minutes in an ethanol concentration of a least 40%, and less than 80% of the active agent after 120 minutes in an ethanol concentration of at least 40%.

Sustained release agents which may be included in the matrix core of the solid pharmaceutical dosage form of the disclosure include, but are not limited to, ethylcellulose, cellulose acetate, vinyl acetate/vinyl chloride copolymers, acrylate/methacrylate copolymers, polyethylene oxide, hydroxypropyl methylcellulose, carrageenan, alginic acid and salts thereof, hydroxyethyl cellulose, hydroxypropyl cellulose, karaya gum, acacia gum, tragacanth gum, locust bean gum, guar gum, sodium carboxymethyl cellulose, methyl cellulose, beeswax, carnauba wax, cetyl alcohol, hydrogenated vegetable oils, and stearyl alcohol. In general, the at least one sustained release agent is present in the composition in an amount of from about 5 wt. % to about 50 wt. %, preferably from about 10 wt. % to about 30 wt. %.

Organic acids or acid salts that assist in maintaining an acidic microenvironment in the matrix core of the solid dosage form can be present in the composition in an amount of from about 0.5 wt. % to about 40 wt. %, preferably from about 1 wt. % to about 20 wt. %. Such acids or salts include triethyl citrate, citric acid, fumaric acid, tartaric acid, adipic acid, glucono delta-lactone, and malic acid.

Polymers that swell under alkaline conditions e.g., at a pH in excess of 5.5 may be incorporated in the matrix core. These polymers include, but are not limited to, acrylic acid copolymers, sodium alginate, carrageenan, alginic acid, pectin, and sodium carboxymethyl cellulose.

Enteric agents include, but are not limited to, cellulose acetate phthalate, hydroxypropyl methylcellulose phthalate, polyvinyl acetate phthalate, methacrylic acid copolymers, cellulose acetate trimellitate, hydroxypropyl methylcellulose acetate, succinate, shellac, and zein.

The composition of the present disclosure may further include other materials such as bulking agents, disintegrating agents, anti-adherents and glidants, lubricants, and binding agents. Bulking agents include, but are not limited to, microcrystalline cellulose (e.g., Avicel®, FMC Corp., Emcocel®, Mendell Inc.), mannitol, xylitol, dicalcium phosphate (e.g. Emcompress, Mendell Inc.) calcium sulfate (e.g. Compactrol, Mendell Inc.) starches, lactose, sucrose (Dipac, Amstar, and Nutab, Ingredient Technology), dextrose (Emdex, Mendell, Inc.), sorbitol, cellulose powder (Elcema, Degussa, and Solka Floe, Mendell, Inc.) The bulking agent may be present in the composition in an amount of from about 5 wt. % to about 90 wt. %, preferably from about 10 wt. % to about 50 wt. %.

Disintegrating agents which may be included in the matrix core of the composition of the disclosure include, but are not limited to, microcrystalline cellulose, starches, crospovidone (e.g. Polyplasdone XL, International Specialty Products.), sodium starch glycolate (Explotab, Mendell Inc.), and crosscarmellose sodium (e.g., Ac-Di-Sol, FMC Corp.). The disintegrating agent may be present in the composition in an amount of from about 0.5 wt. % to about 30 wt %, preferably from about 1 wt. % to about 15 wt. %.

Antiadherents and glidants which may be employed in the matrix core of the composition include, but are not limited to, talc, corn starch, silicon dioxide, sodium lauryl sulfate, and metallic stearates. The antiadherent or glidant may be present in the composition in an amount of from about 0.2 wt. % to about 15 wt. %, preferably from about 0.5 wt. % to about 5 wt. %.

Lubricants which may be employed in the matrix core of the composition include, but are not limited to, magnesium stearate, calcium stearate, sodium stearate, stearic acid, sodium stearyl fumarate, hydrogenated cotton seed oil (sterotex), talc, and waxes, including but not limited to, beeswax, carnuba wax, cetyl alcohol, glyceryl stearate, glyceryl palmitate, glyceryl behenate, hydrogenated vegetable oils, and stearyl alcohol. The lubricant may be present in an amount of from about 0.2 wt. % to about 20 wt. %, preferably from about 0.5 wt. % to about 5 wt. %.

Binding agents which may be employed in the matrix core include, but are not limited to, polyvinyl pyrollidone, starch, methylcellulose, hydroxypropyl methylcellulose, carboxymethyl cellulose, sucrose solution, dextrose solution, acacia, tragacanth and locust bean gum. The binding agent may be present in the composition in an amount of from about 0.2 wt. % to about 10 wt. %, preferably from about 0.5 wt. % to about 5 wt. %.

The compositions of the present disclosure may be made by a direct compression method, or by a wet granulation method. In the direct compression method, the at least one pharmaceutically active agent and other ingredients can be sieved through a stainless steel screen, such as a 40 mesh steel screen. The sieved materials can be charged to a suitable blender, and blended for a suitable period, e.g., 10 minutes with an intensifier bar on for 3 minutes. The blend can be compressed into tablets on a rotary press using appropriate tooling. The compressed tablets are then coated and dried.

In the wet granulation method, the at least one pharmaceutically active agent and other ingredients are granulated with a granulating fluid (e.g., isopropyl alcohol, ethyl alcohol, and water) in a planetary mixer, high shear mixer, or fluidized bed granulator. Binding agents may be contained in the granulating fluid, or may be in the dry mix. The wet granules are dried in an oven or fluidized bed dryer, and then sieved through a suitable screen to obtain free flowing granules. The resulting granules were blended with a suitable lubricant and glidant, and the lubricated granules are compressed into tablets on a rotary press using appropriate tooling. A coating is then applied onto the compressed tablets and dried by a conventional method.

The term “semi-permeable functional film coating” used in surrounding the matrix core of the present formulations can be defined as a coating which is permeable to specific solvents or materials, possibly in one direction. For example, semi-permeable coatings may admit water into the dosage form while preventing the release of the active agent. In the alternative, the semi-permeable membrane may allow release of the active agent while preventing the entry of water or other solvents. Semi-permeable functional film coatings are dissolved more readily than seal coatings, but less readily than permeable coatings, in an acidic environment.

The semi-permeable functional film coating is typically applied to the core as an aqueous dispersion. For present purposes, the term “aqueous dispersion” can be described as dispersed substances in the dispersing agent, water, and can be gas in water (foam), fluid in water (emulsion), or solid in water (suspension). When the dispersed phase is a polymer, it is called a “polymeric dispersion,” and the dispersed phase can be solid, fluid, or in any intermediate condition. The term “latex” is used for colloidal polymer dispersion. The matrix core can be coated with materials used for film coating, i.e., as described in Pharmaceutical Coating Technology (G. Cole (ed.), 1995). Film coating formulations may usually contain the following components: polymer(s), plasticizer(s), colorant(s)/opacifier(s), and vehicle(s).

In a film coating suspension additional quantities of flavors, surfactants, adhesion enhancers can be used, for example, saccharides selected from but not limited to the group consisting of polydextrose, maltodextrin and lactose and waxes. The majority of the polymers used in a film coating can be either cellulose derivatives, such as the cellulose ethers, or acrylic polymers and copolymers. Occasionally encountered can be high molecular weight polyethylene glycols, polyvinyl pyrrolidone, polyvinyl alcohol and waxy materials. Their function usually is to prevent bad mouth feel and/or taste and in some cases degradation, e.g. oxidation of the active ingredients and/or excipients used.

Typical cellulose ethers which may be applied as coatings are hydroxyethylcellulose, hydroxypropylcellulose, hydroxypropylmethylcellulose and methylcellulose. Acrylic polymers comprise a group of synthetic polymers with diverse functionalities. Some of them can be further modified to enhance swelling and permeability by the incorporation of materials such as water soluble cellulose ethers and starches in order to ensure complete disintegration/dissolution of the film.

The commonly used plasticizers can be categorized into three groups: polyols such as glycerol, propylene glycol, macrogols, organic esters such as phthalate esters, dibutyl sebacetate, citrate esters, triacetin, oils/glycerides such as castor oil, acetylated monoglycerides, fractionated coconut oil.

Colorants/opacifiers are classified into several groups: organic dyes and their lakes, inorganic colors, and natural colors. Different materials from each group can also be combined in defined ratios. Film coating suspensions can also be used as ready-to-make preparations which are available on the market such as, for example, Opadry®, Opadry® II, Opadry® AMB, Opadry® fx, Opadry® ns-g, Opadry® NS, Opadry® tm etc.

Although film coating dispersion can be prepared using different solvents such as water, alcohols, ketones, esters, chlorinated hydrocarbons. In various embodiments of the present disclosure, water is always present in the dispersions.

A composition of coating suspension (calculated on dry material) which comprises: 1-99%, preferably 1-95% by weight of polymer, 1-50%, preferably 1-40% by weight of plasticizer, 1-50%, preferably 1-40% by weight of adhesion enhancer, 0.1-20%, preferably 0.1-10% by weight of colorant/opacifier, is particularly preferred.

Conventional equipment for applying a coating, such as conventional coating pans or a Wurster coating system can be used in carrying out embodiments of the present disclosure.

When the pharmaceutically active agent is guanfacine hydrochloride, the composition may be employed in treating an attention deficit disorder, or attention deficit with hyperactivity disorder. The composition including guanfacine hydrochloride is administered to an animal, such as a mammal, including human and non-human primates, in an amount effective to treat the disorders mentioned hereinabove.

The compositions of the present invention may be employed to treat a variety of diseases or disorders.

The disclosure will now be described in more detail by the following non-limiting EXAMPLES. The EXAMPLES are presented to illustrate but a few embodiments of the disclosure. All parts are by weight unless otherwise indicated.

### EXAMPLE 1

| Ingredients | mg/tablet |
|---|---|
| Core Tablet | |
| Part I | |
| Guanfacine HCl | 4.56 |
| HPMC K100M Premium | 97.5 |
| Microcrystalline Cellulose (Avicel PH102) | 44 |
| Lactose (Fast Flo) | 27.8 |
| Compritol 888 ATO | 50 |
| Part II | |
| Ludipress | 32.14 |
| Colloidal Silica (Cab-O-Sil) | 1 |
| Magnesium Stearate | 2 |
| FD&C Yellow #10 Lake HT | 0.6 |
| FD&C Blue #2 Lake HT (11-14%) | 0.4 |
| Total Core Tablet Weight | 260 |
| Semifunctional Coat: | |
| Part III | |
| Clear Opadry (YS-1-7006) | 13 |
| Purified Water, USP | (117) |
| Part IV | |
| Surelease E-7-19010 | (52) |
| Solids Contribution from 25% | 13 |
| Surelease E-7-19010 | |
| Total Coated Tablet Weight | 286 |

### EXAMPLE 2

| Ingredients | Mg/tablet |
|---|---|
| Core Tablet | |
| Part I | |
| Guanfacine HCl | 4.56 |
| HPMC K100M Premium | 97.5 |
| Microcrystalline Cellulose (Avicel PH102) | 43.77 |
| Lactose (Fast Flo) | 28 |
| Compritol 888 ATO | 30 |
| Part II | |
| Ludipress | 42.84 |
| Compritol 888 ATO | 10 |
| Colloidal Silica (Cab-O-Sil) | 1.3 |
| Magnesium Stearate | 1 |
| FD&C Yellow #10 Lake HT | 0.6 |
| FD&C Blue #2 Lake HT (11-14%) | 0.4 |
| Total Core Tablet Weight | 260 |
| Semifunctional Coat: | |
| Part III | |
| Clear Opadry (YS-1-7006) | 13 |
| Purified Water, USP | (117) |
| Part IV | |
| Surelease E-7-19010 | (52) |
| Solids Contribution from 25% | 13 |
| Surelease E-7-19010 | |
| Total Coated Tablet Weight | 286 |

### EXAMPLE 3

| Ingredients | Mg/tablet |
|---|---|
| Core Tablet | |
| Part I | |
| Guanfacine HCl | 4.56 |
| HPMC K100M Premium | 97.5 |
| Microcrystalline Cellulose (Avicel PH102) | 43.77 |
| Lactose (Fast Flo) | 28 |
| Compritol 888 ATO | 30 |
| Part II | |
| Ludipress | 42.84 |
| Compritol 888 ATO | 10 |
| Colloidal Silica (Cab-O-Sil) | 1.3 |
| Magnesium Stearate | 1 |
| FD&C Yellow #10 Lake HT | 0.6 |
| FD&C Blue #2 Lake HT (11-14%) | 0.4 |
| Total Core Tablet Weight | 260 |
| Semifunctional Coat: | |
| Part III | |
| Eudragit RL 30D | (25.5) |
| Solids Contribution from 30% Eudragit RL 30D Dispersion | 7.6 |
| Eudragit RS 30D | (25.5) |
| Solids Contribution from 30% Eudragit RS 30D Dispersion | 7.6 |
| Triethyl Citrate | 3.1 |
| Talc | 7.6 |
| Purified Water, USP | (111.6) |
| Total Coated Tablet Weight | 286 |

To make the respective formulations of Examples 1, 2 and 3, the Part I guanfacine HCl, Hypromellose K100M, microcrystalline cellulose, lactose and Compritol® 888 were roll compacted into hard ribbons and then milled through a Fitz® Mill size reduction apparatus, available from the Fitzpatrick Company of Elmhurst, Ill. USA, to produce the Part I blend. The Part I blend was mixed with the Part II ingredients Ludipress (a directly compressible tabletting excipient comprising lactose monohydrate, povidone K30 and crospovidone, available from BASF), Compritol® 888, if applicable, (a mixture of glyceryl dibehenate and tribehenate and glyceryl behenate, available from Gattefosse-USA), colloidal silica (Cab-O-Sil, available from Cabot Corporation), magnesium stearate and colorants to produce a final blend. The final blend was then compressed into core tablets.

The core tablets were finally coated with the semifunctional coating provided in Part III comprising Surelease®-based aqueous ethylcellulose dispersion available from Colorcon, Inc. of Harleysville, Pa. USA, which provides pH independent uniform drug release, using ethylcellulose as the rate controlling polymer for drug release. The semifunctional coating further contains Clear Opadry® (YS-1-70006), also available from Colorcon, Inc. Clear Opadry® is a suitable component for film coating, which comprises hydroxypropylmethylcellulose and polyethylene glycol.

Alternately, Eudragit® RL 30D or RS 30D (Ammonio Methacrylate Copolymer Dispersion, Type A/Type B-NF) can be used in lieu of Surelease E-7-19010 in the semifunctional dispersion coating. Eudragit® RL 30 D is an aqueous dispersion of Eudragit® RL 100 with 30% dry substance. The water is tested according to the specifications of “Purified Water in bulk” (Ph. Eur.) and according to the specifications for Conductivity of “Purified Water” USP. The dispersion contains 0.25% sorbic acid Ph. Eur./NF as a preservative as well as 0.1% of sodium hydroxide Ph. Eur./NF as an alkalizing agent. Eudragit® RS 30 D is provided as an aqueous dispersion of Eudragit® RS 100 with 30% dry substance. The dispersion contains 0.25% sorbic acid Ph. Eur./NF as a preservative as well as 0.1% of sodium hydroxide Ph. Eur./NF as an alkalizing agent. Eudragit® RS 30 D is described as Type B in the USP/NF monograph quoted above. Eudragit® RL 100 and Eudragit® RS 100 are copolymers of ethyl acrylate, methyl methacrylate and a low content of methacrylic acid ester with quaternary ammonium groups (trimethylammonioethyl methacrylate chloride). The ammonium groups are present as salts and make the polymers permeable. The molar ratio of the monomers of Eudragit RL 100 is approx. 1:2:0.2 and for Eudragit RS 100 is 1:2:0.1. This alternate Eudragit-based coating also contains triethyl citrate and talc, while lacking Opadry®.

Although various exemplary embodiments have been described in detail with particular reference to certain exemplary aspects thereof, it should be understood that the subject matter disclosed herein is capable of other different embodiments, and its details are capable of modifications in various obvious respects. As is readily apparent to those skilled in the art, variations and modifications can be effected while remaining within the spirit and scope of the disclosure. Accordingly, the foregoing disclosure, description, and figures are for illustrative purposes only, and do not in any way limit the invention, which is defined only by the claims.

What is claimed is:

1. A solid pharmaceutical dosage form comprising:
(a) a sustained released matrix core containing i) an at least sparingly water soluble active pharmaceutical ingredient, ii) a hydrophilic polymer, iii) an optional hydrophobic polymer, iv) an optional wax component, and v) an optional diluent; and
(b) a semi-permeable functional film coating directly layered on the core, wherein the dosage form is substantially resistant to dose dumping when administered in the presence of ethanol.

2. (canceled)

3. The dosage form of claim 1 wherein the film coating is substantially insoluble in ethanol.

4. The dosage form of claim 1 wherein the film coating comprises i) a high permeability cationic copolymer derived from esters of acrylic acid and methacrylic acid; and ii) a low permeability cationic copolymer derived from esters of acrylic acid and methacrylic acid.

5. (canceled)

6. (canceled)

7. The dosage form of claim 4 wherein the cationic copolymers are ammonioalkyl methacrylate copolymers and the weight ratio of i) high permability cationic copolymer derived from esters of acrylic acid and methacrylic acid to ii) low permeability cationic copolymer derived from esters of acrylic acid and methacrylic acid ranges from about 0.1 to about 1.0.

8. (canceled)

9. The dosage form of claim 1, wherein the film coating comprises a plasticizer selected from the group consisting of triethyl citrate, triacetin, ethylene vinyl acetate, polyethylene glycol 200 to 8000, glyceryl monostearate, polyvinyl pyrrolidone, diburyl phthalate, dibutyl sebacate, fractioned coconut oil, methylparaben, and a mixture thereof.

10. (canceled)

11. (canceled)

12. The dosage form of claim 1 wherein the film costing comprises an antiadherent selected from the group consisting of glyceryl monostearate, polysorbate 80, talc, and a mixture thereof.

13. (canceled)

14. (canceled)

15. The dosage form of claim 1 wherein the film coating comprises ethylcellulose.

16. The dosage form of claim 15 wherein the film coating further comprises a cellulose ether polymer selected from the group consisting of hydroxymethylcellulose, hydroxypropylcellulose and methylcellulose.

17. (canceled)

18. The dosage form of claim 1 wherein i) the hydrophilic polymer is selected from the group consisting of polyvinylpyrrolidone, hydroxyprophylmethylcellulose, polyethylene oxide, xanthan gum, and a mixture thereof, ii) the optional hydrophobic polymer is selected from the group consisting of ethylcellulose, cellulose acetate, cellulose acetate butyrate, methacrylic acid copolymer, chitosan, and a mixture thereof, iv) the optional wax is selected from the group consisting of glyceryl behenate, carnauba wax, fatty alcohols, hydrogenated vegetable oil, and a mixture thereof, and v) the optional diluent is selected from the group consisting of lactose, microcrystalline cellulose, magnesium stearate, colloidal silica, calcium phosphate dibasic, calcium sulfate, sucrose, mannitol, and a mixture thereof.

19. (canceled)

20. The dosage form of claim 1 wherein the semi-permeable functional film coating ranges from about 5 to about 15 wt. % of the core.

21. (canceled)

22. The dosage form of claim 1 wherein the at least sparingly water soluble active pharmaceutical ingredient is selected from at least one of the group consisting of guanfacine, metoprolol, ropinitole, propranolol, amphetamine, zolpidem, and pharmaceutically acceptable salts thereof.

23. The dosage form of claim 1 wherein the at least sparingly water soluble active pharmaceutical ingredient comprises guarifacine or a pharmaceutically accepted salt thereof.

24. (canceled)

25. The dosage form of claim 1 wherein the release rare of the dosage form in an ethanol concentration of at least 40% is substantially similar to the release rate of the dosage form in an ethanol-free environment.

26. The dosage form of claim 1 wherein less than 60% of the active pharmaceutical ingredient is released after 60 minutes and less than 80% of the active pharmaceutical ingredient is released after 120 minutes, in an ethanol concentration of at least 40%.

27. (canceled)

28. A method for preparing solid pharmaceutical dosage forms comprising:
i) compacting a mixture comprising at least sparingly water soluble active pharmaceutical ingredient, hydroxypropylmethylcellulose, microcrystalline cellulose, lactose, and glyceryl behenate to provide a compacted first blend;
ii) milling the compacted first blend to provide a milled first blend;
iii) mixing the milled first blend with a second blend comprising polyvinylpyrrolidone, crospovidone, lactose, optional glyceryl behenate, colloidal silica, magnesium stearate, and colorant to provide a third blend;
iv) compressing the third blend into core tablets; and
v) applying a semi-permeable functional film coating directly to the core tablets as an aqueous dispersion;
to provide coated tablets substantially resistant to dose dumping when administered in the presence of ethanol.

29. The method of claim 28 wherein due at least sparingly water soluble active pharmaceutical ingredient is selected from at least one of the group consisting of guanfacine, metoprolol, ropinirole, propranolol, amphetamine, zolpidem, and pharmaceutically acceptable salts thereof.

30. The method of claim 28 wherein the film coating comprises i) a high permeability canonic copolymer derived from esters of acrylic acid and methacrylic acid; and ii) a low permeability cationic copolymer derived from esters of acrylic acid and methacrylic acid.

31. The method of claim 28 wherein the film coating comprises ethylcellulose polymer.

32. A method for preparing a solid pharmaceutical dosage which comprises
i) compacting a mixture comprising hydrochloride salt of guanfacine, hydroxpropylmethylcellulose, microcrystalline cellulose, lactose, and glyceryl behenate to provide a compacted first blend;
ii) milling the compacted first blend to provide a milled first blend;
iii) mixing the milled first blend with a second blend comprising polyvinylpyrrolidone, crospovidone, lactose, optional glyceryl behenate, colloidal silica, magnesium stearate, and colorant to provide a third blend;
iv) compressing the third blend into core tablets;
v) applying a semi-permeable functional film coating directly to the core tablets, said semi-permeable functional film coating being substantially insoluble in ethanol, applied as an aqueous dispersion wherein the film coating comprises either (i) a mixture of a high permeability cationic copolymer derived from esters of acrylic acid and methacrylic acid and a low permeability cationic copolymer derived from esters of acrylic acid and methacrylic acid, or (ii) ethylcellulose polymer; and
vi) drying the coated tablets to remove water therefrom;
wherein the resulting dosage form is substantially resistant to dose dumping when administered in the presence of ethanol.

33. The dosage form of claim 1 comprising (a) a sustained released matrix cote comprising hydroxylpropylmethylcellulose and guanfacine hydrochloride; and (b) a semi-permeable functional film coaling comprising ethyl cellulose, hydroxypropylmethylcellulose and polyethylene glycol.
